(19) United States
(12) Patent Application Publication
(10) Pub. No.: US 2006/0200117 A1
(43) Pub. Date: Sep. 7, 2006

(54) Instrument for cryogenic treatments in the medical, paramedical and cosmetic field

(76) Inventor: Erik Hermans, Vollezele (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/535,316

(22) PCT Filed: Nov. 18, 2002
(86) PCT No.: PCT/BE02/00173

§ 371 Date: Mar. 3, 2006

(51) Int. Cl. A61B 18/02

(52) U.S. Cl. 606/25

(57) ABSTRACT

The invention concerns an instrument suited for cryogenic treatments, for the medical or paramedical field as well as for the cosmetic field, comprising a microapplicator having a bore on the order of 20 to 120 μm supplied with a gas stream wherein all the foreign particles above 3 μm and preferably above 1 μm are eliminated. The invention also concerns a microapplicator designed to be used in such an instrument provided with a filter housed in or on said microapplicator, so that each time the latter is replaced, the filter is also replaced.

### FIELD OF THE INVENTION

The present invention concerns an apparatus, which can be used for various cryogenic treatments as well in the medical or paramedical sector as in the cosmetic sector.

### STATE OF THE ART

Numerous dermatological diseases are currently treated with the cryogenic method by using a “jet” of gas, generally nitrous oxide (N₂O) at very low temperature. This cryotechnology is also used in other medical disciplines and in cosmetic fields.

An instrument, among other things, is used, in which a cartridge of liquefied gas, for example N₂O, is introduced, which communicates, generally by perforating a membrane or a protective cap, with a microapplicator in the form of a needle with a very small bore diameter, the whole being contained in a needle cover held by the operator's hand.

Other gas than N₂O (laughing gas) can of course be used.

The gas, contained in a liquid state in the cartridge in the form of gas, is expanded by the release of a “jet” at very low temperature (between about −28° and −90° C.).

The jet is essentially made up of N₂O, partly gaseous and partly liquid.

Two main and almost prohibitive difficulties appear during the implementation of this technique.

Indeed, it seems that impurities, essentially solid microparticles, are also present in the jet and they quickly cause clogging-up of the microapplicator.

The applicant has observed that the impurities are essentially caused by residues from solvents used for preliminary cleaning of the cartridge or particles released during the perforation of the membrane or the protective cap provided therefor on the cartridge.

Other impurities can, for example, come from friction between parts of the instrument's body or with the cartridge, during the introduction of the latter.

It is not excluded that the process of liquid gas production is also a supplementary cause for the observed obstructions due to the presence of impurities in the gas when it is conditioned to cartridges.

Even tiny particles can cause important obstructions taking into account the very small dimension of the bore diameter applied in the microapplicator and the effect of “nucleation” as will be referred to hereafter.

Another observed phenomenon is condensation of moisture contained in the atmosphere in the form of ice, which strongly contributes to the clogging-up depending on the kind of material used for the manufacturing of the microapplicator, due to a phenomenon of “icing”.

This phenomenon of “icing” can be observed especially, for e.g. metallic microapplicators. It also seems that the presence of impurities particles contributes to the “icing” due to the effect of nucleation, i.e. condensation of the atmosphere air under the form of ice on small particles possibly present in the “gaseous” jet.

### AIMS OF THE INVENTION

The present invention aims at avoiding the described disadvantages of apparatuses according to the state of the art, in particular to avoid their clogging-up and to allow better performance by easing their use.

The present invention also aims at suggesting a process to interrupt a gas flow in particular in certain medical apparatuses.

Finally, the present invention aims at suggesting various possibilities of use in the medical, paramedical or cosmetic sector.

### SUMMARY OF THE INVENTION

The applicant has observed that the optimal operating conditions rely on the principle that a microapplicator must show a bore diameter of about 20 to 120 μm and that a constant and regular flow of the liquefied gas contained in the cartridge can only be obtained if the foreign particles possibly present in the microapplicator's flow are such that those bigger than 3 μm and preferably bigger than 1 μm are eliminated. Means suitable for eliminating particles comprised between 1 and 100 μm, but preferably between 3 and 60 μm according to the said bore diameter are preferably used.

This can be obtained by using a condensed gas that has undergone a preliminary purification to eliminate solid materials.

However, in most of the cases, in practice it can be observed that even resorting to especially purified condensed gas does not necessarily solve the problem and according to a supplementary characteristic of the invention, which corresponds to a preferred embodiment, it is foreseen that the microapplicator is supplied with a removable filter that retains particles bigger than 1 μm, preferably 1.5 μm.

Different kinds of filters can be used such as porous ceramic, cellulose material, etc.

To avoid that the filter progressively clogs up by accumulating particle deposits during the repetitive use of several successive cartridges in the same equipment, according to a particularly preferred embodiment of the invention the filter can be replaced after 1 to 20 replacements of the gas cartridge. It may be advantageous that the replacement of a cartridge automatically leads to the replacement of the filter with a new filter, avoiding the clogging-up of the microapplicator.

The applicant has also endeavoured to solve observed difficulties as a result of the “icing”, i.e. the sealing-off of the microapplicator with ice coming from the atmospheric moisture. He has noticed that the use of materials, in particular synthetic materials such as polycarbonate or a resin such as PEEK, strongly reduces this phenomenon to the extent that possible phenomena of icing do not lead to a clogging-up.

Other materials, with suitable thermal conductivity characteristics e.g. glass, can suit to that end. The material must of course be selected to resist at the same time at very low temperatures observed during treatment and at high temperatures necessary for sterilisation.

Moreover, the invention also aims at providing a disposable microapplicator suitable for the apparatus of cryogenic treatment as described.

Examples of uses in gynaecological, urological or dermatological domains can be mentioned.

Regarding the use of the apparatus, it has been observed that it is advantageous to have the possibility to interrupt the gas flow during the use, so that the user can realise a short and precise treatment avoiding in particular to treat healthy tissues.

The fact that the cartridge is not always empty when it is replaced can be dangerous for the user. At that end, it is foreseen to permit evacuation of the gas.

### SHORT DESCRIPTION OF THE DRAWINGS

FIG. 1 represents a sectional view of the apparatus according to the invention.

FIGS. 2A to 2D represent various positions of a valve comprised in the apparatus.

FIG. 3 represents a detailed view of the head of the apparatus illustrating the position of the filter.

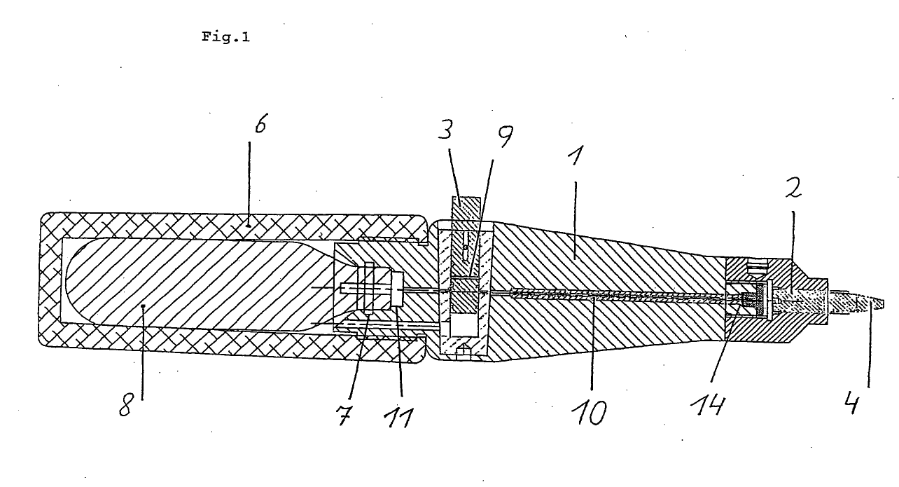

The same reference numerals are used for identical constituent elements in the three figures.

### DESCRIPTION OF A PREFERRED EMBODIMENT OF THE INVENTION

The apparatus for cryogenic treatment comprises a body 1. On this body, a microapplicator 2, protected with a cover when not in use, is fixed in an impervious but removable manner.

On the back of the body 1, an insertion tube 6 is mounted. It can receive a condensed gas cartridge 8. An O-ring 7 creates an impervious connection between the cartridge and the body 1. In use, when the cartridge 8 is in place, the mounting of the insertion tube 6 on the body 1 causes the perforation of a protective cap 11 that closes up the cartridge 8 and permits so the communication through the pipe 10 between the cartridge 8 and the microapplicator 2 through a valve 3.

The microapplicator 2 has a bore diameter 4 of between about 20 to 120 μm, preferably of between about 35 to 80 μm. It is attached to a longitudinal pipe 10 with filter 14 interposed therebetween, preferably maintained in place on the front removable part and more specific at the back of the microapplicator 2.

The adjustment of the gas flow coming from the cartridge 8 is realised by any device providing this function.

A valve 3 is provided between this device and the microapplicator 2, which is perpendicular to the pipe 10. The valve can have three distinct possible positions under the effect of an electrical or mechanical control. In the first position, a longitudinal pipe 9 provides the communication so that the gas flows from the device of the flow adjustment to the microapplicator 2. In a second position the gas flow is blocked. The third position permits that the residual gas, which is present in the cartridge 8, can escape.

In the preferred embodiment of the FIGS. 2A to 2D, the valve 3 is represented closed, half-open, open and in the position for removal of residual gas from the cartridge.

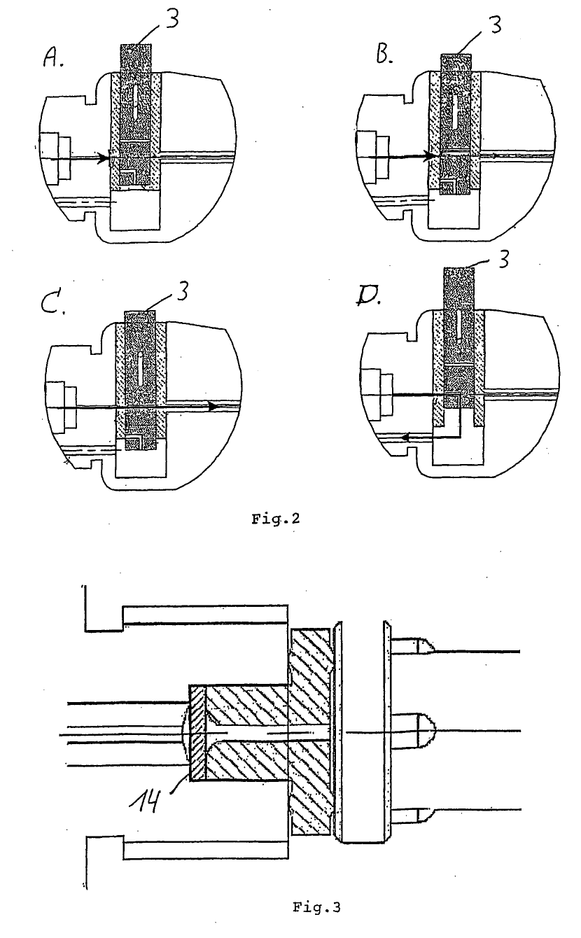

Before arriving at the microapplicator 2, the gas must pass through a filter 14 detailed in FIG. 3.

Using a filter 14 disposed on the microapplicator 2 is a particular advantageous solution since the replacement of the filter is easily realised with the dismassembly of the microapplicator 2, optionally together with the disassembly of the insertion tube 6 for the replacement of the cartridge 8. In this case, it is not necessary (though useful) to use an especially purified condensed gas for the cartridge 8. The filter can be in the form of a disc receivable in a slot at the end of pipe 10 that is located in or on the microapplicator and maintained in place by a capsule. This capsule is perforated and can be separated from or consolidated with filter 14. The latter solution facilitates the correct placement of the filter 14, the replacement of the filter being executed in this case with the replacement of the capsule.

Although particularly advantageous embodiments of the invention have been described, alternatives can be provided for the described equipment, while staying within the scope of the claims.

The invention is not in particular limited to the indicated condensed gas, nor to the forms or dimensions of the suggested equipment.

1. An apparatus for cryogenic treatments for use in the medical or paramedical field as well as for the cosmetic field, comprising a microapplicator having a bore diameter of 20 to 120 μm supplied with a gas flow from which all foreign particles bigger than 3 μm have been eliminated.

2. The apparatus of claim 1, additionally comprising a cartridge of purified condensed gas from which all solid materials have been eliminated.

3. The of claim 1, additionally comprising a cartridge containing N₂O.

4. The apparatus of claim 1, wherein to microapplicator comprises a replaceable filter arranged to retain particles superior to 3 μm.

5. The apparatus of claim 4, wherein the microapplicator comprises a replaceable filter arranged to retain particles between 1 and 100 μm in function of the said bore diameter.

6. The apparatus of claim 4, wherein the filter is located in or on the microapplicator.

7. The apparatus of claim 6, wherein the microapplicator consists of a synthetic material or a resin to reduce the phenomena of icing and the clogging-up of said microapplicator.

8. The apparatus of claim 1, further comprising:
a pipe;
a flow regulator for regulation of the flow in the said pipe;
a valve, said valve being disposed perpendicularly to said pipe between said device and the said microapplicator and having three distinct possible positions under the effect of a mechanical or electrical control, comprising:
a first position where a longitudinal pipe is created, which allows the flow of gas from the device to the microapplicator%
a second position where the gas flow is blocked:
a third position which permits to the gas present in the cartridge to escape.

9. A process for interrupting a gaseous flow in a medical devices comprising:
providing a cylindrical valve comprising a transverse pipe which permits gas flow from a cartridge to a microapplicator, said valve being perpendicular to the direction of the gas flows and
providing a mechanical or electrical actuator to permit upward and downward movement of said valve and providing O-rings for imperviousness.

10. The process of claim 9, wherein the cylindrical valve comprises a vent, which allows escape of residual gas.

11. A microapplicator for the apparatus of claim 1, wherein the imcroapplicator it comprises a mounted removable filter.

12. A method for cosmetic treatment and/or dermatological treatment of the skin, comprising use of the apparatus of claim 1.

13. A method for gynaecological or urological treatment comprising use of the apparatus of claim 1.

14. The apparatus of claim 1, wherein all foreign particles bigger than 1 μm have been eliminated from the gas flow.

15. The apparatus of claim 1, wherein the microapplicator comprises a replaceable filter arranged to retain particles larger than 1 μm.

16. The apparatus of claim 4, wherein the microapplicator comprises a replaceable filter arranged to retain particles between 3 and 60 μm in function of the said bore diameter.

17. The apparatus of claim 7, wherein said synthetic material is a polycarbonate.

18. The apparatus of claim 7, wherein said resin is PEEK.
